(19) United States
(12) Patent Application Publication
(10) Pub. No.: US 2017/0072072 A1
(43) Pub. Date: Mar. 16, 2017

(54) PH SENSITIVE FLUORESCENT COMPOUNDS AND METHODS FOR TUMOR DETECTION

(71) Applicant: THE METHODIST HOSPITAL SYSTEM, Houston, TX (US)

(72) Inventors: Ching-Hsuan Tung, Houston, TX (US); Jianjun Qi, Houston, TX (US)

(21) Appl. No.: 15/126,064

(22) PCT Filed: Mar. 17, 2015
(86) PCT No.: PCT/US15/20983

§ 371 Date: Sep. 14, 2016

Related U.S. Application Data
(60) Provisional application No. 61/954,764, filed on Mar. 18, 2014.

(51) Int. Cl. A61K 49/00; C09B 23/08

(52) U.S. Cl. CPC A61K 49/0032; C09B 23/08

(57) ABSTRACT

pH-sensitive fluorescent compounds that can generate a detectable signal for the relatively small changes in proton concentration associated with cancerous tissue are described. Methods of making and using the compounds to help detect cancerous tissue are also disclosed.

### ACKNOWLEDGEMENT OF GOVERNMENT SUPPORT

This invention was made with government support under Grants No. CA135312 and GM094880 awarded by the National Institutes of Health. The government has certain rights in this invention.

### BACKGROUND

The tumor microenvironment constitutes a complex system that includes tumor cells, immune cells, fibroblasts, vascular structures, and an extracellular matrix rich in signaling molecules (Hanahan et al., Cell 144:646 (2011); Schiavoni et al., Frontiers Oncol 3:90 (2013). Neoplastic lesions are not self-sufficient in their propagation and require otherwise normal supporting cells and proliferative or protective factors within this dynamic milieu. A key feature of this microenvironment is the mildly acidic condition generated by the altered metabolism, also known as the “Warburg effect” of tumors, and relative hypoxia (Estrella et al., Cancer Research 73:1524 (2013); Webb et al., Nature Reviews. Cancer 11:671 (2011); Gatenby et al., Nature Rev Cancer 4:891 (2004)). In addition, the intracellular pH (pHᵢ) and extracellular pH (pHₑ) in cancerous cells are distinct from normal cells (FIG. 1A). In non-neoplastic cells the pH, and pHₑ are 7.2 and 7.5, respectively; but in cancer cells, the pH, is 7.5 and the pHₑ is about 6.4-7.1 (Webb et al., Id.; Choi et al., J Pathology 230:350 (2013); Calcinotto et al., Cancer Res 72:2746 (2012); De Milito et al., Int J Cancer 127:207 (2010)). To maintain their rapid growth and proliferation, cancer cells have a higher need for energy which is partially satisfied by greater reliance on alternate, albeit less efficient, metabolic pathways. Under aerobic conditions, cancer cells metabolize glucose to lactic acid, instead of pyruvate which enters the Krebs cycle observed in normal cells. In combination with relatively poor perfusion, the lactic acid generated by this process lowers the pHₑ. Recent studies indicate that by maintaining a relatively low pH in their microenvironment cancer cells can escape immune detection (Webb et al., Id.; Calcinotto et al. Id.). In addition, the acidic environment promotes or facilitates the action of many proteases that are involved in tissue remodeling and tumor invasion (Rozhin et al., Cancer Res 54:6517 (1994); Busco et al., FASEB J24:3903 (2010)). Indeed, areas of low pHₑ often observed at tumor boundaries correspond to high proteolytic activity (Id.), supporting an intimate role for extracellular acidification in several hallmarks of cancer (Hanahan et al., Id.).

### SUMMARY

In accordance with the purposes of the disclosed materials, compounds, compositions, articles, devices, and methods, as embodied and broadly described herein, the disclosed subject matter relates to compositions and methods of making and using the compositions. In a specific aspect, the subject matter disclosed herein relates to pH-sensitive fluorescent compounds that can generate a detectable signal for the relatively small changes in proton concentration that occur with cancerous tissue. These compounds can be non-toxic, biocompatible, cell permeable, and generate a signal that is detectable using relatively common equipment adaptable for surgical or clinical applications. Methods of using the disclosed compounds to detect cancerous tissue in vivo, e.g., during a tumor resection procedure are also disclosed.

Additional advantages of the disclosed subject matter will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying Figures, which are incorporated in and constitute a part of this specification, illustrate several aspects of the invention and together with the description serve to explain the principles of the invention.

FIG. 1A is a cartoon showing the intracellular pH (pHᵢ) and extracellular pH (pHₑ) in tumor and normal tissues. FIG. 1B is a schematic diagram of the fluorescence activation of CypH-1. The arrows indicate electrons.

FIG. 2A is a graph showing the pH response of CypH-1 measured with Ex=740 nm and Em=785 nm. The pKₐ is 5.3. FIG. 2B are bright field and NIRF images of CypH-1 in the pH range 5-8. FIG. 2C are micrographs showing the intracellular distribution of CypH-1. SKOV3 cells were incubated with CypH-1 (1 μM) for 20 min, washed and then incubated with each organelle tracker (0.5 μM) for 1 h. The images show CypH-1 signal (red) co-registered with the signal of lyso-tracker and mito-tracker, but not ER tracker.

FIG. 3A is a group of in vivo images of CypH-1 in an ovarian cancer model. Nude mice with GFP positive SKOV3 tumor were imaged 3 h after IP injection of CypH-1. Green fluorescence indicated the location of the GFP⁺ tumors, which were also found NIRF positive. FIG. 3B is a group of ex-vivo imaging of individual organs, and FIG. 3C is a graph showing the quantification of the tumor to organ ratio (TOR). T: tumor, In: intestine, K: kidney, L: liver, M: muscle, Sp: spleen, and St: stomach.

FIG. 4A contains macroscopic images showing different signal intensity in tumors. The peripheral areas giving strong acetic signals might not have the highest number of GFP tumor cells; therefore, the signals don't always match. FIG. 4B is a group of microgrpahs showing the histological correlation of the fluorescent signal of tumor and normal tissue (4×). The pH signal was only seen on the edge of the tumor (top row), but not in normal tumor (bottom row). T: tumor, Sp: spleen.

FIG. 5A is a graph showing the pH response of CypH-1S measured with Ex=740 nm and Em=785 nm. CypH-1S is an aqueous soluble derivative of CypH-1. FIG. 5B are bright field and NIRF images of CypH-1S in the pH range 5-8.

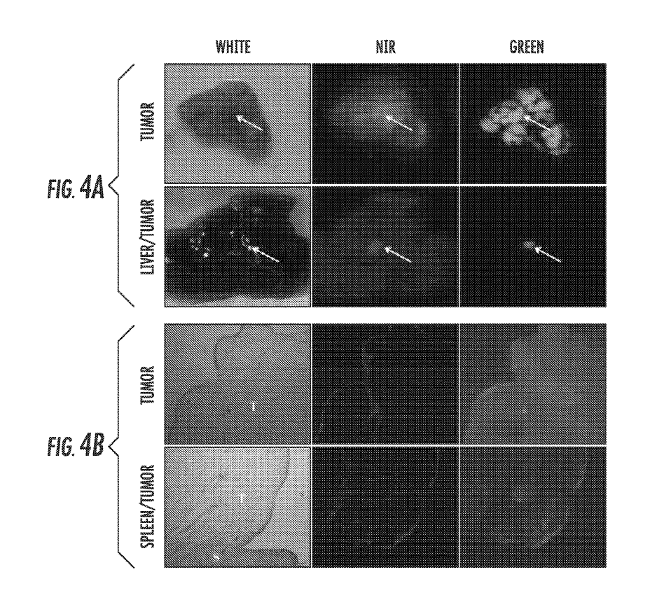

FIG. 6 is a graph showing the pH response of CypH-1L measured with Ex=740 nm and Em=785 nm. CypH-1L is a derivative of CypH-1 with a functional group for conjugation.

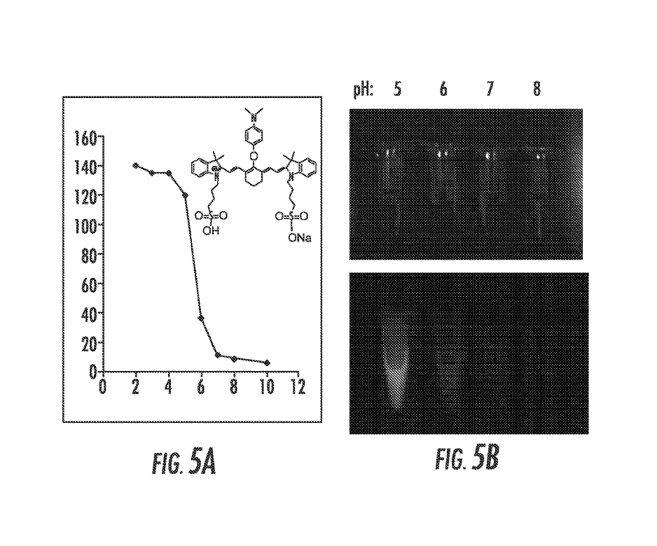

FIG. 7 is a graph showing the normalized absorption (black) and emission (grey) spectra of CypH-1 at pH=4 phosphate buffer, Exₘₐₓ=760 nm; Emₘₐₓ=777 nm.

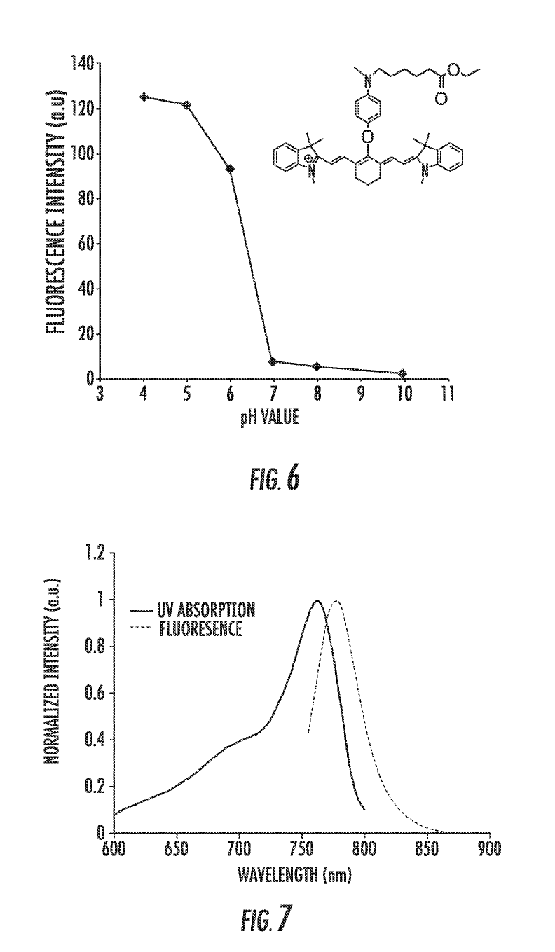

FIG. 8 is a graph showing the quantum yield measurement of CypH-1 at pH 4.0 (green) and pH 7.4 (black) using indocyanine green (red) in DMSO as a reference (Φ=0.106). Φ_{pH4}=Φ_{Standard}[G_{radx}/G_{rads}][η_{H2O}/η_{DMSO}]²=0.106[4×108/5×108][1.33/1.479]²=0.06857 Φ_{pH7.4}=Φ_{Standard}[Gradx/G_{rads}][η_{H2O}/ηDMSO]²=0.106[0.1×108/5×108][1.33/1.479]²=0.001714.

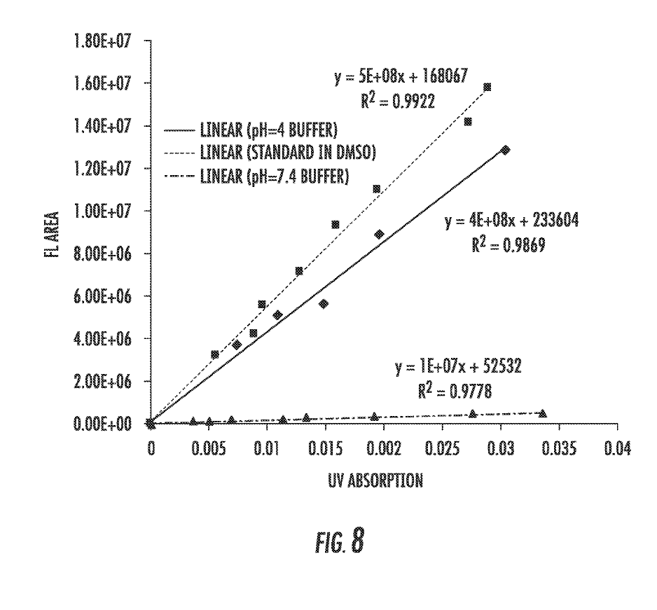

FIG. 9A is a pair of fluorescence images performed at 5 min post-dye spray using a NIRF imaging system on procured human tumor sectioned into three portions and treated with the vehicle control (O) and two concentrations of CypH-1 (2 and 5 μM). FIG. 9B is a graph showing the relative signal intensities quantified using dedicated analysis software. FIG. 9C is a photomicrograph of a tumor sample sprayed with the 2 μM CypH-1 and later processed and stained with H/E. The focus of tumor located centrally measures approximately 1.1 cm. 20×.

FIG. 10A is a white light image of SKOV3 tumor (GFP+, pale tissue) on spleen (dark tissue) in CypH-1 probe solution (1 μM). FIG. 10B is a GFP image of SKOV3 tumor (GFP+) on spleen in CypH-1 probe solution (1 μEM). FIG. 10C is a NIRF image of SKOV3 tumor (GFP+) on spleen in CypH-1 probe solution (1 μEM) acquired at 0 minutes. FIG. 10D is a NIRF image of of SKOV3 tumor (GFP+) on spleen in CypH-1 probe solution (1 μM) acquired at 3 min without washing.

### DETAILED DESCRIPTION

The compounds, compositions, articles, devices, and methods described herein can be understood more readily by reference to the following detailed description of specific aspects of the disclosed subject matter and the Examples and Figures.

Before the present compounds, compositions, articles, devices, and methods are disclosed and described it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

When ranges of values are disclosed, and the notation “from n₁ . . . to n₂” is used, where n₁ and n₂ are the numbers, then unless otherwise specified, this notation is intended to include the numbers themselves and the range between them. This range can be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, for example 1 to 3, 1 to 4, 1 to 5, 1 to 6, 1 to 7, 1 to 8, 1 to 9, 1 to 10, or 1 to 15 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as—OZ¹ where Z¹ is alkyl as defined above.

The term “alkenyl” as used herein is a hydrocarbon group of from 2 to 24 carbon atoms, for example, 2 to 5, 2 to 10, 2 to 15, or 2 to 20 carbon atoms, with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, sulfo-oxo, sulfonyl, sulfone, sulfoxide, or thiol, as described below.

The term “alkynyl” as used herein is a hydrocarbon group of 2 to 24 carbon atoms, for example 2 to 5, 2 to 10, 2 to 15, or 2 to 20 carbon atoms, with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “carbonyl as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. Throughout this specification “C(O)” or “CO” is a short hand notation for C═O.

The term “carbamate” or “carbamyl” as used herein, alone or in combination, refers to an ester of carbamic acid (—NZ¹C(O)O—) which can be attached to the parent molecular moiety from either the nitrogen or acid end, and which can be optionally substituted as defined herein. The term “O-carbamyl” as used herein, alone or in combination, refers to a —OC(O)NRR′ group; and the term “N-carbamyl” as used herein, alone or in combination, refers to a Z¹OC(O)NZ²— group, where Z¹ and Z² can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide”, “halo”, or “halogen” as used herein refers to the fluorine, chlorine, bromine, and iodine.

The term “hydroxyl” as used herein is represented by the formula —OH.

The term “nitro” as used herein is represented by the formula —NO₂.

The term “nitrile” as used herein is represented by the formula —N₃.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above. The term “sulfonylamino” or “sulfonamide” as used herein is represented by the formula —S(O)₂NH—. The term “sulfonate” is used herein to refer to —SO₃⁻, which is a deprotonated sulfonic acid moiety —SO₃H. “Sufonate” can also refer to the sulfonate salt, e.g., the Li, Na, K, Ca, Mg, NH₄, salt even though the particular counterion may not be specified. The term “sulfinyl” as used herein refers to S(O)Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above. The term “sulfo-oxo” is used herein to generally refer to sulfonyl, sulfonylamino, sulfonate, sulfonic acid, and sulfinyl groups.

The term “thiol” as used herein is represented by the formula —SH. The term “thio” or “sulfanyl” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising a sufonyl group,” the sulfonyl group can be incorporated within the backbone of the alkyl group. Alternatively, the sulfonyl group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compounds

Disclosed herein are pH-sensitive fluorescent compounds that can augment the detection of tumors by targeting the acidic tumor microenvironment. Since acidic microenvironment is a common factor of most tumors, the disclosed compounds can be used in conjunction with a wide array of tumors. It has been demonstrated herein that CypH-1 is non-fluorescent in normal tissues but fluoresces when it is taken up by neoplastic lesions. The pH-triggered activation can permit high signal to background ratios of lesions as small as 1 mm without the need for a clearance procedure to remove any excess compound, in essence representing a homogeneous in vivo labeling process. These features are well suited for rapid clinical translation in the form of an intraoperative system to augment the detection of tiny metastatic implants important for optimal cytoreductive surgery in malignancies such as ovarian cancer (Bristow et al., J Clin Oncol 20:1248 (2002); Eisenkop et al., Gynecol Oncol 69:103 (1998)). CypH-1 can be intraperitoneally or topically administered to an area of suspected neoplastic lesions prior to or even during surgery, without the need for intravenous administration, reducing the potential for systemic toxicity.

Disclosed herein are compounds that can be membrane permeable and are fluorogenic. The fluorogenic properties are dependent on the pH of tumors. In certain aspects, disclosed herein are pH sensitive fluorescent compounds that have Formula I:

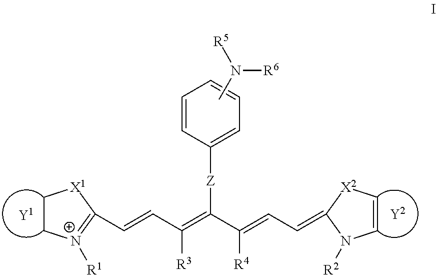

wherein Z is O or S;
Y¹ and Y² can be, independent of one another, cyclic groups, e.g., cycloalkyl, heterocycloalkyl, aryl, or heteroaryl, any of which are optionally substituted with sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol;
X¹ and X² can be, independent of one another, O, S, NH, C(CH₃)₂, or C═CH₂;
R¹ and R² can be, independent of one another, H or alkyl, alkenyl, alkynyl, cycloalkyl, heterocycloalkyl, cycloalkenyl, or heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol;
R³ and R⁴ can be, independent of one another, H or alkyl, alkenyl, alkynyl, cycloalkyl, heterocycloalkyl, cycloalkenyl, or heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; or
R³ and R⁴ can be joined together and form a cycolpentenyl or cyclohexenyl ring, which is optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; and
R⁵ and R⁶ can be, independent of one another, H or alkyl, alkenyl, alkynyl, cycloalkyl, heterocycloalkyl, cycloalkenyl, heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; or
R⁵ and R⁶ can be joined together and form a piperadine, piperazine, or pyrrolidine ring, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol;
or pharmaceutically acceptable salts thereof.

In certain examples of Formula I, the amine substituent, (R⁵)(R⁶)N—, is in the ortho position. In other examples of Formula I, the amine substituent, (R⁵)(R⁶)N—, is in the meta position. In still other examples of Formula I, amine substituent, (R⁵)(R⁶)N—, is in the para position.

In a subgenus of Formula I, the amine substituent, (R⁵)(R⁶)N—, is in the para position and Z is O, as shown below in Formula I-A.

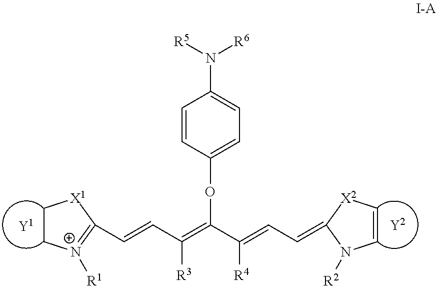

In Formula I, and unless expressly stated to the contrary, Formula I-A as well, Y¹ and Y² can be, independent of one another, aryl or heteroaryl, any of which are optionally substituted with sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In still further examples of Formula I, Y¹ and Y² can have, independent of one another, one of the following formulas.

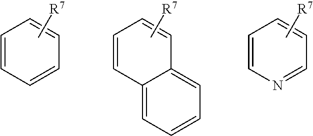

wherein R⁷ can be hydrogen, sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol.

In certain other examples of Formula I, Y¹ and/or Y² can be a heteroaryl group selected from pyrrolyl, pyrrolinyl, imidazolyl, pyrazolyl, pyridyl, pyrimidinyl, pyrazinyl, pyridazinyl, imidazolyl, triazinyl, triazolyl, tetrazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, or xanthenyl, any of which can be substituted with sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol.

In preferred examples of Formula I, Y¹ and Y² can be unsubstituted phenyl or naphtyl. In specific examples of Formula I, X¹ and X² can be, independent of one another, O or C(CH₃)₂; and in preferred examples, X¹ and X² are both C(CH₃)₂.

In specific examples of Formula I, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples, R¹ and R² can be, independent of one another, alkyl substituted with sulfonic acid or sulfonate. In more preferred examples, R¹ and R² can be C₁-C₆ alkyl, and in particular methyl.

In some examples of Formula I, R³ and R⁴ can be, independent of one another, H, alkyl, or alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples of Formula I, R³ and R⁴ can be joined together and form a cycolpentenyl or cyclohexenyl ring, more preferably a cyclohexenyl ring.

In some examples of Formula I, R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. For example, R⁵ and R⁶ can be, independent of one another, alkyl optionally substituted with amine, amido, alkylester, carbonyl, carboxylate, carbamyl, or hydroxyl, preferably an alkylester. In preferred examples of Formula I, R⁵ and R⁶ can both be C₁-C₆ alkyl, for example, methyl.

In certain examples of Formula I, R¹, R², R⁵, and R⁶ are each C₁-C₆ alkyl, for example, methyl.

Also, disclosed herein are pH sensitive fluorescent compounds that have Formula II:

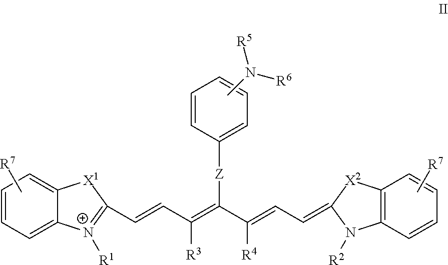

wherein Z, X¹, X², R¹, R², R³, R⁴, R⁵, R⁶, and R⁷ are as defined hereinabove for Formula I. For example, disclose herein are compounds of Formula II wherein X¹ and X² can be, independent of one another, O or C(CH₃)₂. In preferred examples of Formula II, X¹ and X² are both C(CH₃)₂. R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples of Formula II, R¹ and R² can be, independent of one another, alkyl substituted with sulfonic acid or sulfonate. In more preferred examples of Formula II, R¹ and R² can be C₁-C₆ alkyl, and in particular methyl. R³ and R⁴ can be joined together and form a cycolpentenyl or cyclohexenyl ring, more preferably a cyclohexenyl ring. R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. For example, R⁵ and R⁶ can be, independent of one another, alkyl optionally substituted with amine, amido, alkylester, carbonyl, carboxylate, carbamyl, or hydroxyl, preferably an alkylester. In preferred examples of Formula II, R⁵ and R⁶ can both be C₁-C₆ alkyl, for example, methyl. And R⁷ can be hydrogen, sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol.

In certain examples of Formula II, the amine substituent, (R⁵)(R⁶)N—, is in the ortho position. In other examples of Formula II, the amine substituent, (R⁵)(R⁶)N—, is in the meta position. In still other examples of Formula II, amine substituent, (R⁵)(R⁶)N—, is in the para position. In a subgenus of Formula II, the amine substituent, (R⁵)(R⁶)N—, is in the para position and Z is O, as shown below in Formula II-A.

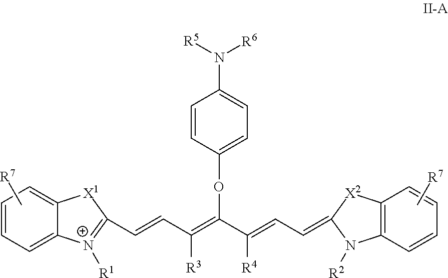

In more specific examples, disclosed herein are pH sensitive fluorescent compounds that have Formula III, including pharmaceutically acceptable salts thereof:

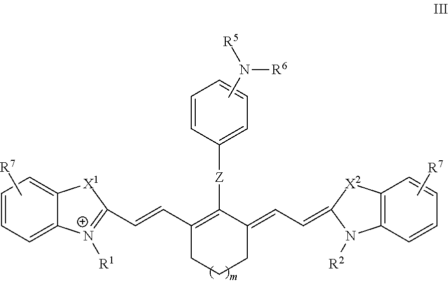

wherein Z, X¹, X², R¹, R², R⁵, R⁶, and R⁷ are as defined hereinabove for formulas I and II, and m is 1 or 2. In another example of Formula III, Z is O and the amine substituent, (R⁵)(R⁶)N—, is in the para position.

In more specific examples, disclosed herein are pH sensitive fluorescent compounds that have Formula IV, including pharmaceutically acceptable salts thereof:

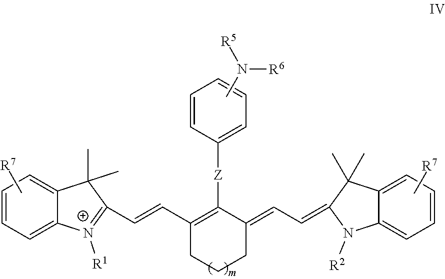

wherein Z, R¹, R², R⁵, R⁶, and R⁷ are as defined hereinabove for Formulas I and II, and m is 1 or 2. In one example of Formula IV, the amine substituent, (R⁵)(R⁶)N—, is in the para position.

In more specific examples, disclosed herein are pH sensitive fluorescent compounds that have Formula V, including pharmaceutically acceptable salts thereof:

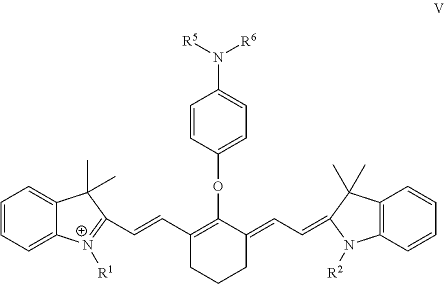

wherein R¹, R², R⁵, and R⁶ are as defined hereinabove for Formulas I and II. For example, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples, R¹ and R² can be, independent of one another, alkyl substituted with sulfonic acid or sulfonate. In more preferred examples, R¹ and R² can be C₁-C₆ alkyl, and in particular methyl. R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. For example, R⁵ and R⁶ can be, independent of one another, alkyl optionally substituted with amine, amido, alkylester, carbonyl, carboxylate, carbamyl, or hydroxyl, preferably an alkylester. In preferred examples, R⁵ and R⁶ can both be C₁-C₆ alkyl, for example, methyl. In certain examples, R¹, R², R⁵, and R⁶ are each C₁-C₆ alkyl, for example, methyl.

In specific examples, the disclosed compounds can be have one of the following formulas,

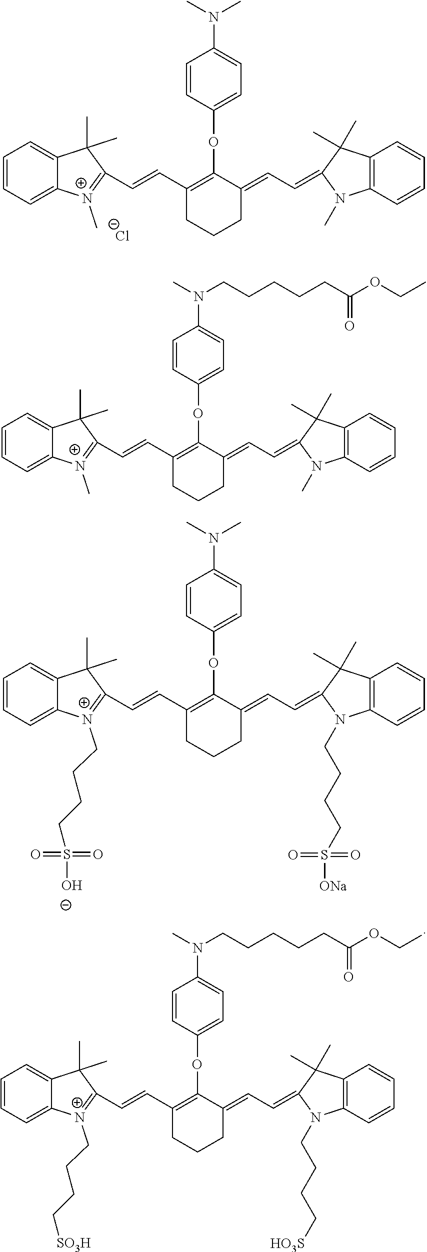

Examples where the O-aryl group is replaced by S-aryl are also disclosed.

The disclosed compounds are not pH insensitive. The disclosed compounds are not hydrophobic dyes, since they can cause high background signals by nonspecific membrane binding. In some examples, the disclosed compounds do not give high fluorescent signals in lysosome.

As noted the disclosed compounds of can include pharmaceutically acceptable salts. As used herein, “pharmaceutically acceptable salts” represents salts or zwitterionic forms of the compounds disclosed herein which are water or oil-soluble or dispersible and pharmaceutically acceptable. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Examples of acids which can be employed to form therapeutically acceptable addition salts include inorganic acids such as hydrochloric, hydrobromic, sulfuric, and phosphoric, and organic acids such as oxalic, maleic, succinic, and citric. In preferred examples, the chloride salts of the disclosed compounds are used. Compositions and Formulations

While it can be possible for disclosed compounds to be administered as the neat compound, it is also possible to present them as a pharmaceutical formulation. Accordingly, provided herein are pharmaceutical formulations which comprise one or more of the disclosed compounds together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art; e.g., in Remington: The Science and Practice of Pharmacy, 21^{st} Ed., Gennaro, Ed., Lippencott Williams & Wilkins (2003). The compositions and formulations disclosed herein can be manufactured in any manner known in the art, e.g., by means of conventional mixing, dissolving, granulating, dragee-making, levigating, emulsifying, encapsulating, entrapping or compression processes.

A compound as disclosed herein can be incorporated into a variety of formulations for therapeutic administration, including solid, semi-solid, or liquid forms. The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route can depend upon for example the condition and disorder of the recipient. The formulations can conveniently be presented in unit dosage form and can be prepared by any of the methods well known in the art of pharmacy. Typically, these methods include the step of bringing into association a compound or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Certain compounds disclosed herein can be administered topically, that is by non-systemic administration. This includes the application of a compound disclosed herein externally to the epidermis or the buccal cavity and the instillation of such a compound into the ear, eye and nose, such that the compound does not significantly enter the blood stream. Formulations suitable for topical administration include liquid or semi-liquid preparations suitable for penetration through the skin or tissue to the site of interest as gels, liniments, lotions, creams, ointments or pastes. In preferred examples, the formulations comprise a pharmaceutically acceptable solvent. Examples of pharmaceutically acceptable solvents include sterile aqueous ethanol, a polyol (for example, glycerol, propylene glycol, liquid polyethylene glycols, and the like), vegetable oils, nontoxic glyceryl esters, and suitable mixtures thereof. Thickeners such as synthetic polymers, fatty acids, fatty acid salts and esters, fatty alcohols, modified celluloses or modified mineral materials can also be employed with pharmaceutically acceptable solvents to form spreadable pastes, gels, ointments, soaps, and the like, for application directly to the skin of the user. In specific examples, the pharmaceutically acceptable solvent can be a PBS solution with optional cosolvents like DMSO. In specific examples, the formulation can be a spray formulation.

### Methods of Making

The disclosed compounds can be synthesized by nucleophilic substitution of the fluorgenic substrate A with the aminophenolic compound B, as shown in the Scheme 1.

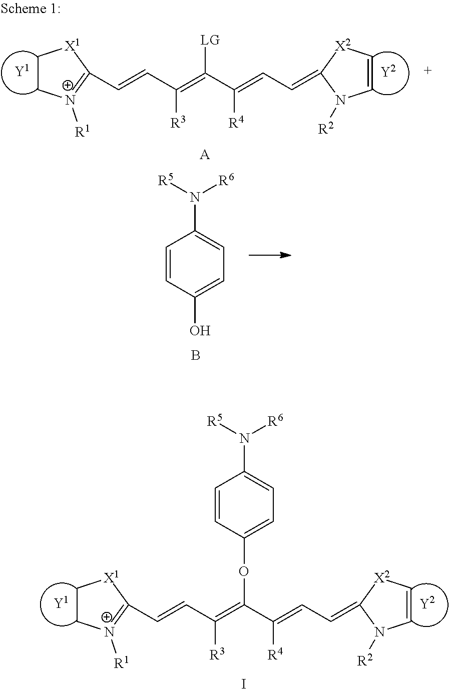

where LG is a leaving group like a Cl, Br, I, tosylate, mesylste, or triflate, and the other substituents are as defined above. This reaction can be performed with a strong base like sodium or potassium hydride, lithium aluminum hydride, or an organolithium base like t-butyl lithium. The reaction can be done in a suitable solvent like DMF, THF, DMSO, or CH₂Cl₂ at room temperature.

The fluorgenic substrate A can be synthesized from commercially available, or easily synthesizable, heterocylic amines as shown in Scheme 2:

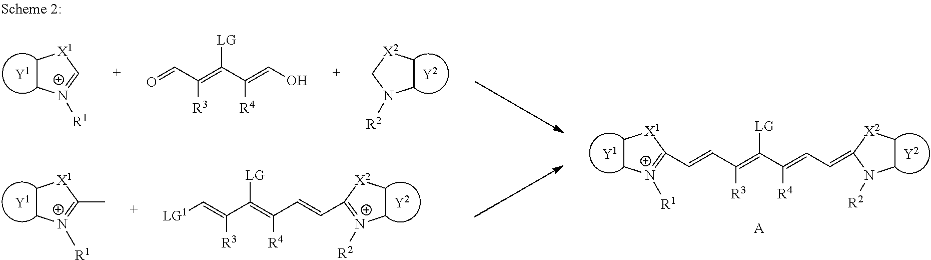

Each route can be initiated by the addition of base, e.g., potassium acetate or pyridine. The top route is best suited for preparing compounds when the two hetrocyclic amines are the same. The heterocyclic amines can be prepared by alkylating the appropriate heterocyclic precursor as shown in Schemes 3 and 4:

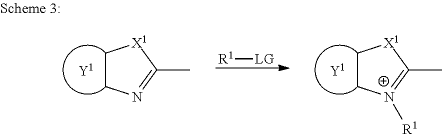

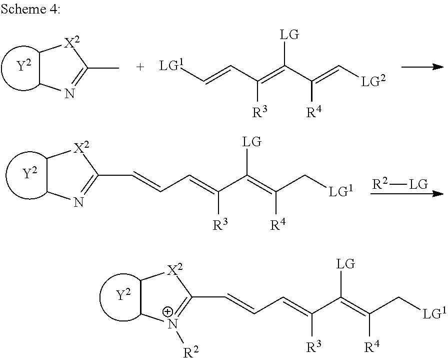

### Methods of Using

The acidic micro-environment of tumors is known to facilitate cancer proliferation. This localized acid-base imbalance can be imaged with the disclosed pH-sensitive fluorogenic compounds. The disclosed compounds that are non-fluorescent at neutral pH, but fluoresces under mildly acidic conditions, can be viewed with a near infrared maximum emission wavelength.

Different tumor types can demonstrate varying degrees of pH changes in their microenvironments (Gerweck et al., Cancer Res 56:1194 (1996); Engin et al., Int J Hyperthermia 11:211 (1995)); however, the disclosed compounds can detect physiologically relevant acidic conditions created by a combination of extracellular and intracellular changes. Thus, the disclosed compounds and compositions containing them can be used as imaging agents to visualize cancerous tissue, e.g., tumors. In one aspect, disclosed herein is a method of detecting cancer in vivo comprising administering a compound as disclosed herein to an individual and detecting a fluorescent signal. The disclosed compounds can be administered to an individual by intraperitoneal injection before a tumor resection procedure or during the procedure by a topical spray. A region of interest in the individual can be imaged using a fluorescence reflectance imaging system (such as the F-Pro from Broker), which is fitted with multiple band pass filters for excitation and emission.

In recent years, many new fluorescence imaging systems, such as endoscopes (Hsiung et al., Nat Med 14:454 (2008); Funovics et al., Mol Imaging 2:350 (2003)), wide-field video cameras (Knapp et al., European urology 52:1700 (2007); van Dam et al., Nat Med 17:1315 (2011)), and goggles (Liu et al., Surgery 149:689 (2011); Wang et al., J Biomed Opt 15:020509 (2010)), have been developed. Any of these systems can be used to detect the fluorescent signal, or lack thereof, in an individual. Further, the development of the fluorescence can be followed using a near infrared video camera (e.g., Fluoptics).

In one example, disclosed is a method of detecting colon cancer. Most colonic cancers develop from adenomatous polyps, a pre-malignant lesion. If untreated, some of them could turn into malignant lesions (carcinoma), invading surrounding tissues and eventually spreading systemically. Identification and removal of colonic adenomatous polyps during endoscopy is the most reliable method of reducing the incidence of colorectal cancer. However current gastrointestinal imaging largely relies on simple endoscopic examination of the lesions, a limited technique for the detection and staging of the disease. It has been found that these so call “benign adenomatous polyps” behaved similar to the malignant adenocarcinoma in protease activity (Marten et al., Gastroenterology 122:406-14 (2002)) and recruitment of folate positive inflammatory macrophages (Chen et al., Mol Imaging 4:67-74 (2005)). Low pH, 6.93 ±0.08, in these tissues also has been reported (Engin et al., Int J Hyperthermia 11:211-6 (1995)). As such, the disclosed compounds can be administered to an individual during a colonoscopy and the fluoresce of the compound measured to detect early dysplastic adenomas, and thereby greatly enhance the diagnosis of cancer.

The compounds disclosed herein can be used to detect a variety of other cancers since the pH difference between normal tissue and many types of cancer can be detected by the change of fluoresce of the disclosed compounds. Examples of cancer types detectable by the compounds and compositions disclosed herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples included cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

In other examples, the disclosed compounds can be administered during surgery to resect a tumor. The disclosed compounds can result in robust fluorescence signal of discrete neoplastic lesions with millimeter range resolution. Moreover, their fluorescence signal is strikingly enhanced at peripheral regions of tumors at the microscopic level, indicating a sharp pH gradient at the tumor/normal tissue interface. As such, the disclosed compounds can be used as a wash or spray in the region of the tumor resection before the resection; this can highlight the area and allow better identification of cancerous tissue to be resected. Alternatively or additionally, the disclosed compounds can be administered during or after the resection; this can highlight the area and allow better identification of any remaining cancerous tissue left behind. In this way, cancer cells that were not excised and still present in the body can be identified. Alternatively, the disclosed compositions can be administered around the time of surgery (peri-operative), before surgery (pre-operative), or after surgery (post-operatively). The compounds can be administered a week, 2-5 days, 1-3 days, 1-18 hours, 1-12 hours, 1-6 hours, or less than an hour before or after tumor resection surgery.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

In the following Examples, the human SKOV3 cervical cancer cell line was obtained from the American Type Culture Collection (ATCC, Manassas, Va.). The luciferase transfected GFP+ SKOV3 (SKOV3/GFP-Luc) cells were purchased from Cell Biolabs (San Diego, Calif.). The cell lines were maintained in McCoy's 5A Medium, supplemented with 10% fetal bovine serum, 1% antibiotics (penicillinstreptomycin) at 37° C. under 5% CO₂. Lyso Tracker Red, Mito Tracker Green, ER Tracker Blue and Hank's balanced salt solution (HBSS) were purchased from Life Technologies, CA. Fluorescent microscopic images of live cells were acquired using an inverted fluorescence microscope (Olympus 81×; Tokyo, Japan). The fluorescence image of CypH-1 was collected through a cy7 filter set (excitation 675-745 nm, emission 765-855 nm), Lyso Tracker Red was obtained through the TRITC band pass filter set (excitation: 510-550 nm, emission: 573-648 nm), Mito Tracker Green was obtained through the FITC filter set (excitation: 450-490 nm, emission: 500-550 nm), and ER Tracker Blue was taken via the DAPI filter set (excitation: 325-375 nm, emission: 435-485 nm).

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

### Synthesis and Characterization of Dye CypH-1

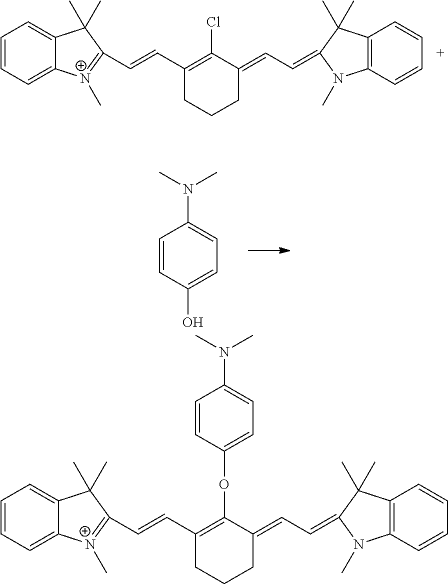

A near infrared (NIR) fluorogenic cyanine pH-sensitive (CypH) dye was synthesized by placing an amino group near the conjugated double bonds on a Cy7 analog. Specifically, 4-(dimethylamino)phenol (57 mg, 0.42 mmol, JACS 2011, 133, 16970) in DMF (5 ml) was reacted with sodium hydride (17 mg) at RT for 15 min. Commercially available IR-775 (100 mg, 0.19 mmol, Aldrich, Dye content ˜90%) was added slowly and stirred at RT overnight. The reaction was extracted with dichloromethane (DCM), and washed with brine. The product in DCM layer was purified by silica gel column using DCM/MeOH (MeOH 0-10%) as an elution solvent. The yield was 57 mg, 48%. TLC: DCM/MeOH=10/1, Rf=0.25. 1H-NMR (300 MHz, CDCl3): 7.87 (2H, d, J=14.1 Hz), 7.71 (2H, d, J=7.8 Hz), 7.41-7.35 (2H, m), 7.31 (2H, d, J=6.3 Hz), 7.25 (2H, d, J=7.5Hz), 7.21-7.16 (2H, m), 7.09 (2H, d, J=7.8 Hz), 6.00 (2H, d, J=14.1 Hz), 3.58 (6H, s), 3.13 (6H, s), 2.75-2.65 (4H, m), 2.05-2.00 (2H,m), 1.36 (12H, s). 13C-NMR (125 MHz, MeOD): 172.77, 163.72, 158.83, 142.55, 141.99, 140.78, 139.21, 128.72, 125.44, 122.28, 122.08, 116.23, 110.26, 100.12, 49.02, 45.67, 31.22, 27.63, 24.18, 20.92. MS: 584 (M+). Bioconjugate Chem. 22:2227-36 (2011).

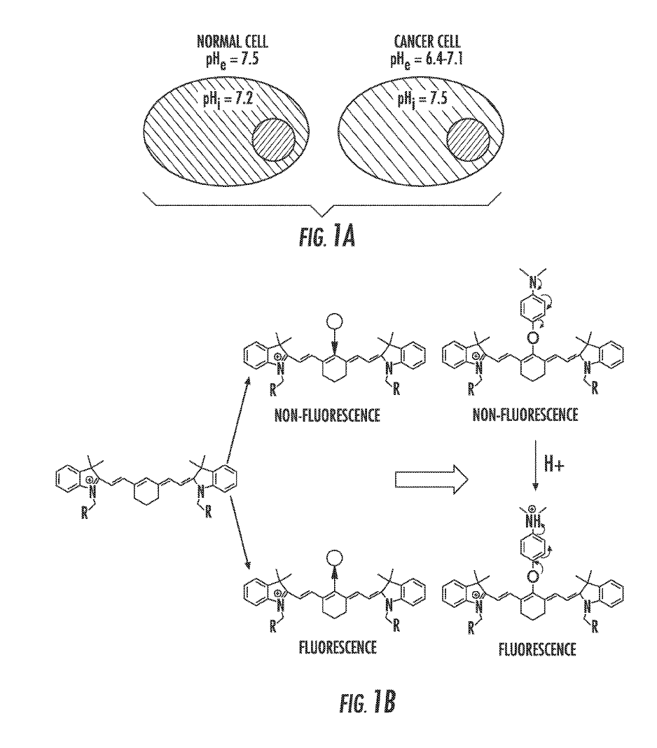

CypH-1 altered fluorescence intensity through a photoinduced electron transfer (PeT) mechanism, which involves signal quenching by the lone pair electrons from the amine group (FIG. 1B). When the amines are protonated under acidic conditions, fluorescence is de-quenched resulting in a strong signal (FIG. 1B).

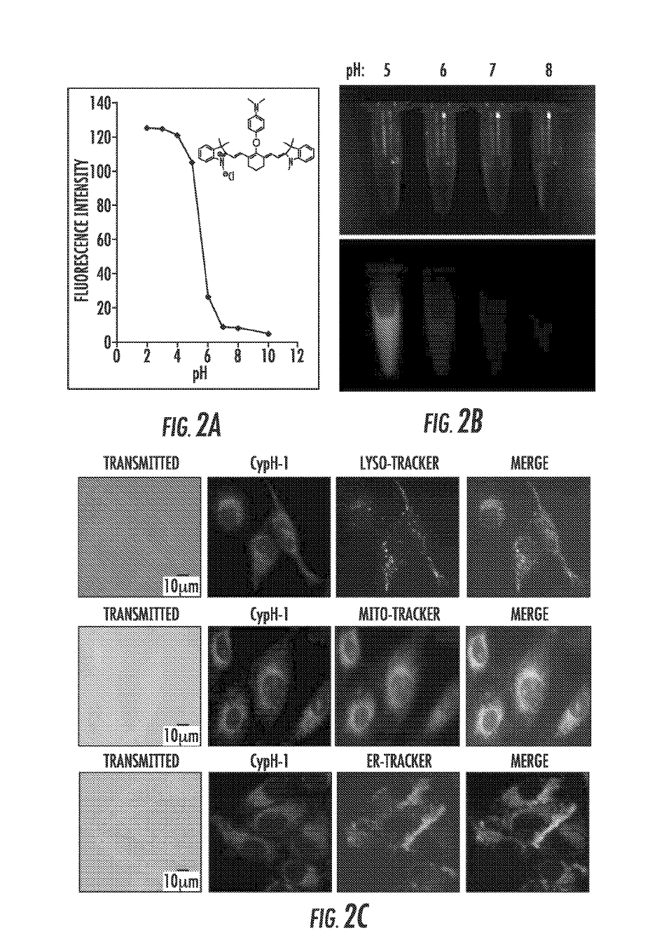

It was found that CypH-1 has almost no fluorescence at neutral and basic conditions (≧7.0), but fluoresces brightly when the solution is acidic (≦pH 5.0) (FIG. 2A). The excitation and emission maxima of CypH-1 are 760 and 777 nm, respectively. The quantum yield (Φ) of CypH-1 at pH 7.4 and 4.0 are 0.17% and 6.9%, respectively, representing up a 40-fold difference in fluorescence signal intensity. Near infrared fluorescence (NIRF) imaging confirmed intense fluorescence signal at pH 5.0, less intense fluorescence at pH 6.0, and no detectable signal at pH 7.0 or 8.0 (FIG. 2B).

### Example 2

### CypH-1 In Vitro Testing

The intracellular localization properties of CypH-1 were determined by using an immortalized human ovarian adenocarcinoma cell line, SKOV-3, for cell uptake and live cell fluorescence microscopy. Specifically, SKOV3 (5×10³/well) were seeded in a 96-well plate for 20 hrs. Cells were incubated in phenol red free DMEM (Hyclone, Logan , Utah) with 10% FBS and 1% antibiotics with the presence of CypH-1 (0.5 μM) for 30 mins at 37° C. Cells were then washed using HBSS (4×), followed by incubation with Lyso Tracker Red (0.5 μM), Mito Tracker (0.5 μM) or ER Tracker (0.5 μM) for 20-30 min. The concentration and labeling condition of each tracker was suggested by the manufacturer. Cells were then washed with HBSS (4×) and incubated with fresh complete medium (phenol-red free) for 20 min before fluorescence imaging.

As shown in FIG. 2C intracellular fluorescence was detected clearly with negligible background signal. The intracellular distribution of CypH-1 was compared to three organelles—lysosomes, mitochondria, and the endoplasmic reticulum (ER)—using commercially available labeling reagents, LysoTracker, MitoTracker, and ER-Tracker, respectively. CypH-1 precisely co-localized with LysoTracker (FIG. 2C, top row) strongly supporting its rapid lysosomal uptake and also fully explains CypH-l′s activation in the low pH environment of this organelle (pH˜5). Interestingly, CypH-1 was also activated in mitochondria, again strongly supported by the known acidic compartments involved in the chemiosmotic production of ATP by this organelle (FIG. 2C, second row) (Bonnet et al., Cancer Cell 11:37 (2007)). In contrast to the observed lysosome and mitochondria localization, CypH-1 signal was not observed in the ER or the cell nucleus. Since CypH-1 has no fluorescence in neutral pH, after incubation, the cells could be imaged immediately without any washes.

### Example 3

### CypH-1 In Vivo Testing

All animal studies were performed in compliance with the approved animal protocols and guidelines of Institutional Animal Care and Use Committee of Houston Methodist Research Institute. BALB/c Nu/Nu female nude mice (n=15) were bought from Charles River (Wilmington, Mass.). A xenograft murine model generated by the intraperitoneal (IP) inoculation of SKOV-3 cells that have been modified to express luciferase and green fluorescence protein (GFP). Specifically, SKOV3/GFP-Luc (5×10⁶) cells were injecting intraperitoneally (IP) into the abdomen of 5 weeks old nude mice. Tumor growth was followed by luciferase imaging. After two weeks of initial tumor inoculation, CypH-1 (20 nmol) dissolved in 2.5% DMSO (100 μL) was administrated IP Animals were sacrificed in 3 hrs and, without any washing procedures or subsequent handling, imaged under GFP and NIR channel using the IVIS 200 system (PerkinElmer, Waltham, Mass.). The GFP signal is used to locate tumors. The GFP single was collected using 445-490 nm excitation and 515-575 nm emission filter set, while CypH-1 signal was collected using 710-760 nm excitation and 810-875 nm emission filter set. Signal to background ratio was performed selecting specific ROI (Region of Interested) using IVIS software.

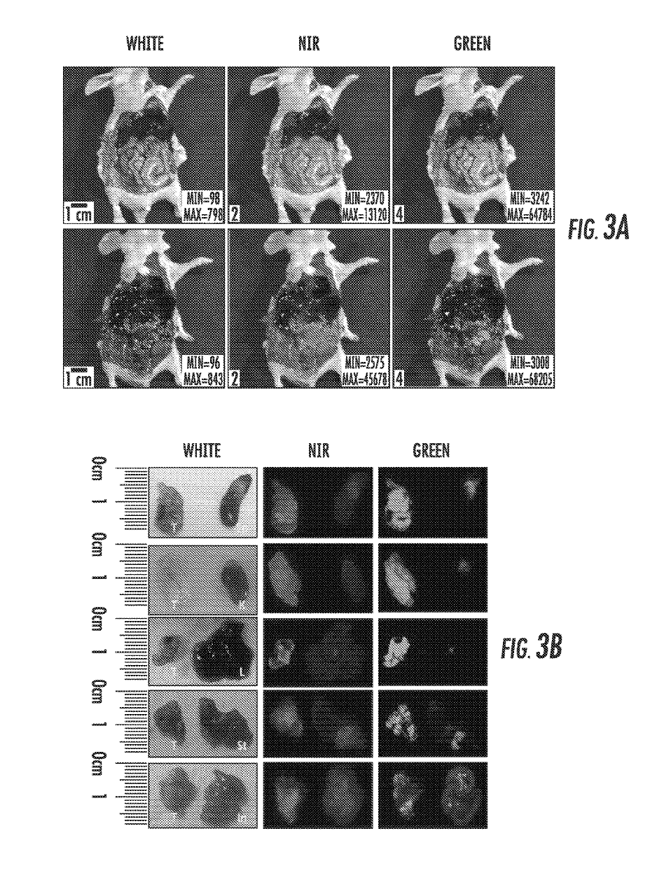

Since all tumors expressed GFP the green fluorescence signal corresponded to grossly visible tumors seen under white light (FIG. 3A). Fluorescence imaging using the near-infrared filters revealed precise signal co-localization with GFP-positive lesions (FIG. 3A). To further characterize additional disseminated lesions of varying size, tumors and several intraabdominal major organs (kidney, liver, spleen, stomach, intestine, and tumors) were resected and imaged with in a zoomed filed of view using a separate fluorescent imaging system (Maestro, CRI/Perkin Elmer). The GFP signal was collected using 445-490 nm excitation and 515 nm long pass emission filter set. The CypH-1 signal was collected using 710-760 nm excitation and 800 nm long pass emission filter set. Tumor to Organ Ratio (TOR) was determined by measuring mean fluorescence intensities for each tissue and tumor. Statistical analysis was performed using student-t test method. Differences with p values of less than or equal to 0.01 were considered statistically significant. Again, there is a good agreement between the NIRF and GFP signals indicating of viable tumors (FIG. 3B).

Both gross in situ and ex vivo imaging show overall favorable low background NIRF signal following the intraperitoneal administration of CypH-1 in the absence of a washing procedure before imaging. The relatively low background tissue signal permitted high tumor contrast even for tiny lesions that are marginally visible in white light (FIG. 3B). Fluorescence signal ratio of tumor to normal muscle was 24.5:1 (FIG. 3C). When compared to normal organs high contrast was observed. The ratio in kidney, liver, spleen, and stomach were 9, 4, 4, and 4, respectively. The only exception was intestine, where the tumor signal is only slightly higher than the background (ratio=1.3). There is a diffusely low signal from the gastrointestinal tract which may be due to nonspecific fluorescence from the diet ingested by the animals.

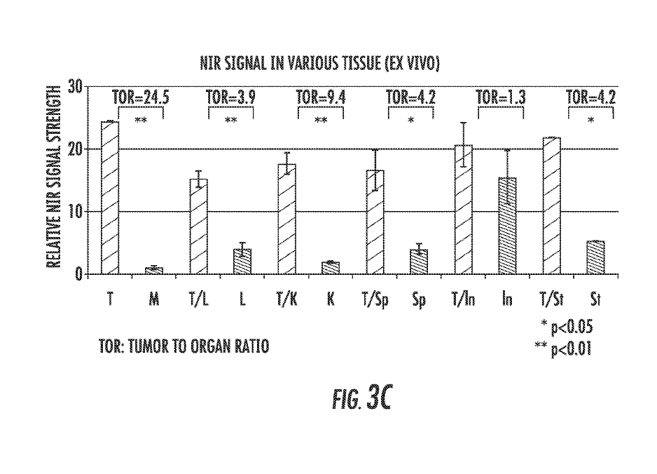

Interestingly, at higher magnification the distribution of NIRF signal of discrete tumors and tiny metastatic implants did not always match exactly in areas of focal intensity. Heterogeneous NIRF signal intensity is more prominent in either a peripheral or central distribution relative to the GFP signal which suggests areas of greater acidity (lower pH) that do not necessarily reflect the number of viable tumor cells (FIG. 4A). Further analysis of a tiny metastatic implant in the liver revealed an interesting distribution of CypH-1 signal that was most intense in the periphery of the lesion that measured approximately 1 mm in diameter (FIG. 4A). This peripheral distribution pattern was confirmed at the microscopic level. As shown in FIG. 4A the NIRF signal was seen on the surface of a resected tumor (FIG. 4B, upper panel) and at the interface between tumor and spleen (FIG. 4B, lower panel). The sharp interface strongly supports the pH difference between the tumor and normal tissues. The distribution of the pH signal is completely different from the GFP signal, which is distributed throughout the whole tumor. Since CypH-1 was administered intraperitoneally it is possible that the peripheral signal observed in the tissues reflects the degree of the absorption or penetration.

### Example 4

### Human Tissue Study

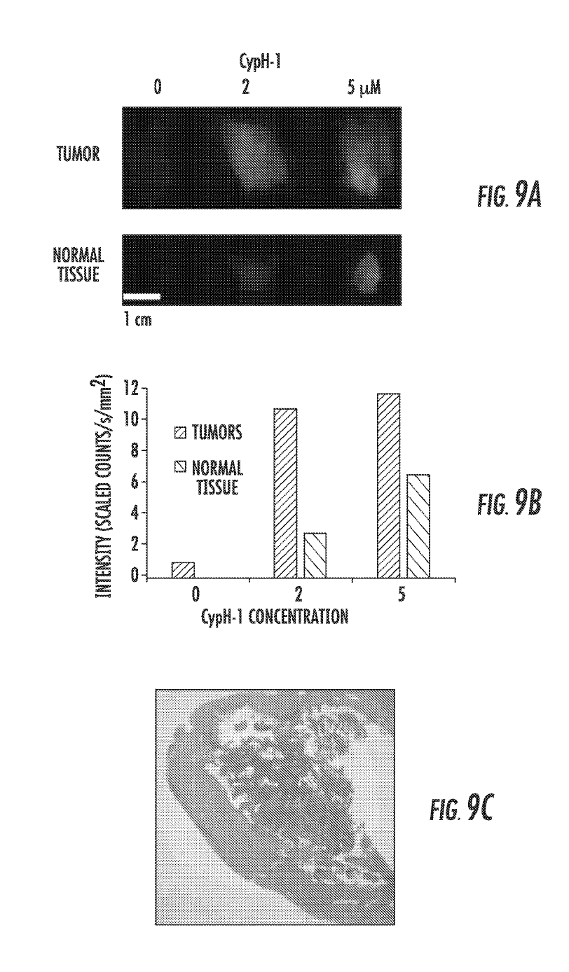

Procured human ovarian tumor and normal tissue samples were sectioned into three portions. Two concentrations of CypH-1 and a vehicle control were sprayed onto all tissue samples. Thereafter the tissue samples were placed in the CCD camera for imaging with a total elapsed time of 5 minutes after dye application (FIG. 9A). Semiquantitative analysis of the fluorescence intensity reveals ˜3.8 fold greater signal in the tumor relative to normal tissue at the 2 μM CypH-1 concentration (FIG. 9B). The tumor to normal signal ratio was reduced at 5 μM concentration to ˜1.8. Tumor samples were sent for histochemical processing. A representative photomicrograph is shown in FIG. 9C corresponding to the tumor section that was exposed to CypH-1 at 2 μM. The photomicrograph shown in FIG. 9C demonstrates a focus of tumor approximately 1.1 cm in greatest dimension, in good agreement with the fluorescence image.

### Example 5

### Mouse Spleen Study

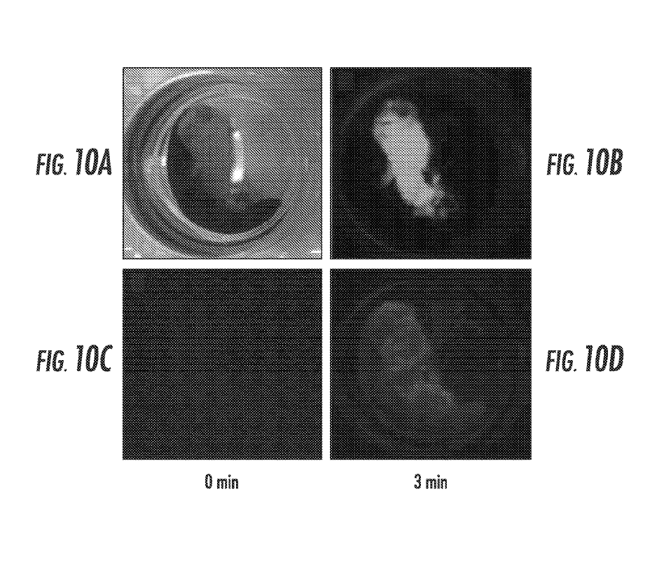

To study the time needed for signal development, a mouse spleen with a tumor was submerged into a CypH-1 solution (1 μM), and images of the tissue were acquired at different time intervals without wash. It was found that in less than 3 minutes, the tumor, which expresses GFP, was clearly visible in the NIRF image with exquisitely high resolution detail (compare FIGS. 10C and 10D). The fluorescent signal reached a plateau in less than 10 minutes. This quick development of the fluorescence is expected, as the pH change is an instantaneous reaction. The tumor signal was developed rapidly within a few minutes after in contact with the tumor tissue.

### Specific Embodiments

In specific embodiments, disclosed herein are pH sensitive fluorescent compounds having Formula I:

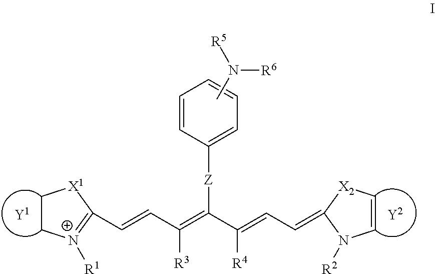

wherein Z can be O or S; Y¹ and Y² can be, independent of one another, cycloalkyl, heterocycloalkyl, aryl, or heteroaryl, any of which are optionally substituted with sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; X¹ and X² can be, independent of one another, O, S, NH, C(CH₃)₂, or C═CH₂; R¹ and R² can be, independent of one another, H or alkyl, alkenyl, alkynyl, cycloalkyl, heterocycloalkyl, cycloalkenyl, or heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; R³ and R⁴ can be, independent of one another, H or alkyl, alkenyl, alkynyl, cycloalkyl, heterocycloalkyl, cycloalkenyl, or heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; or R³ and R⁴ can be joined together and form a cycolpentenyl or cyclohexenyl ring, which is optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; and R⁵ and R⁶ can be, independent of one another, H or alkyl, alkenyl, alkynyl, cycloalkyl, heterocycloalkyl, cycloalkenyl, heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; or R⁵ and R⁶ can be joined together and form a piperadine, piperazine, or pyrrolidine ring, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; or pharmaceutically acceptable salts thereof. Specific examples of compounds with Formula I are compounds that have the formula:

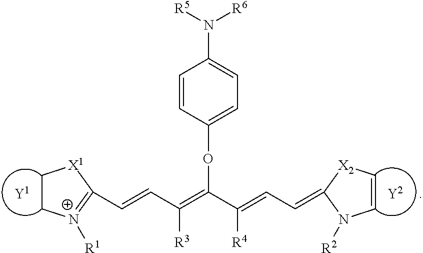

In any of the disclosed formula, Y¹ and Y² can be, independent of one another, aryl or heteroaryl, any of which are optionally substituted with sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, Y¹ and Y² can be, independent of one another, one of the following formulas.

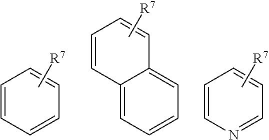

wherein R⁸ can be hydrogen, sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples, Y¹ and Y² can be unsubstituted phenyl or naphtyl. In any of the disclosed formula, X¹ and X² can be, independent of one another, O or C(CH₃)₂. In preferred examples, X¹ and X² can both be C(CH₃)₂. In any of the disclosed formula, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, R¹ and R² can be, independent of one another, alkyl substituted with sulfonic acid or sulfonate. In a preferred example, R¹ and R² can be C₁-C₆ alkyl. In any of the disclosed formula, R³ and R⁴ can be, independent of one another, H, alkyl, or alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples, R³ and R⁴ can be joined together and form a cycolpentenyl or cyclohexenyl ring. In any of the disclosed formula, R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In certain examples, R⁵ and R⁶ can be, independent of one another, alkyl optionally substituted with amine, amido, alkylester, carbonyl, carboxylate, carbamyl, or hydroxyl. In preferred examples, R⁵ and R⁶ can both be C₁-C₆ alkyl. In a preferred example, R¹, R², R⁵, and R⁶ can each be C₁-C₆ alkyl.

In another specific embodiment, disclosed are compounds having Formula II:

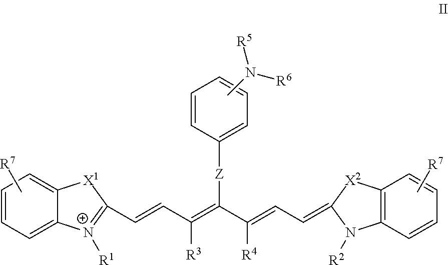

wherein R⁷ can be hydrogen, sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In any of these disclosed formula, X¹ and X² can be, independent of one another, O or C(CH₃)₂. In preferred examples, X¹ and X² can both be C(CH₃)₂. In any of these disclosed formula, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, R¹ and R² can be, independent of one another, alkyl substituted with sulfonic acid or sulfonate. In a preferred example, R¹ and R² can be C₁-C₆ alkyl. In any of these disclosed formula, R³ and R⁴ can be, independent of one another, H, alkyl, or alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In preferred examples, R³ and R⁴ can be joined together and form a cycolpentenyl or cyclohexenyl ring. In any of the disclosed formula, R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In certain examples, R⁵ and R⁶ can be, independent of one another, alkyl optionally substituted with amine, amido, alkylester, carbonyl, carboxylate, carbamyl, or hydroxyl. In preferred examples, R⁵ and R⁶ can both be C₁-C₆ alkyl. In a preferred example, R¹, R², R⁵, and R⁶ can each be C₁-C₆ alkyl. In any of these disclosed formula, Z can be S. In preferred examples, Z can be O.

In another specific embodiment, disclosed are compounds having Formula III:

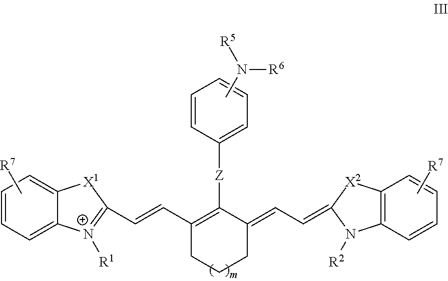

wherein IV is hydrogen, sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; and m is 1 or 2. In any of the disclosed formula, X¹ and X² can be, independent of one another, O or C(CH₃)₂. In preferred examples, X¹ and X² can both be C(CH₃)₂. In any of these disclosed formula, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, R¹ and R² can be, independent of one another, alkyl substituted with sulfonic acid or sulfonate. In a preferred example, R¹ and R² can be C₁-C₆ alkyl. In any of these disclosed formula, R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In certain examples, R⁵ and R⁶ can be, independent of one another, alkyl optionally substituted with amine, amido, alkylester, carbonyl, carboxylate, carbamyl, or hydroxyl. In preferred examples, R⁵ and R⁶ can both be C₁-C₆ alkyl. In a preferred example, R¹, R², R⁵, and R⁶ can each be C₁-C₆ alkyl. In any of the disclosed formula, Z can be S. In preferred examples, Z can be O.

In another specific embodiment, disclosed are compounds having Formula IV:

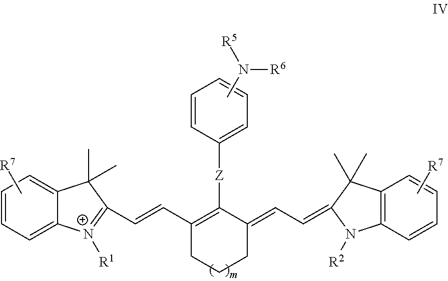

wherein R⁷ is hydrogen, sulfonic acid, sulfonate, alkylsulfonic acid, alkylsulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; and m is 1 or 2. In any of the disclosed formula, X¹ and X² can be, independent of one another, O or C(CH₃)₂. In preferred examples, X¹ and X² can both be C(CH₃)₂. In any of these disclosed formula, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, R¹ and R² can be, independent of one another, alkyl substituted with sulfonic acid or sulfonate. In a preferred example, R¹ and R² can be C₁-C₆ alkyl. In any of these disclosed formula, R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In certain examples, R⁵ and R⁶ can be, independent of one another, alkyl optionally substituted with amine, amido, alkylester, carbonyl, carboxylate, carbamyl, or hydroxyl. In preferred examples, R⁵ and R⁶ can both be C₁-C₆ alkyl. In a preferred example, R¹, R², R⁵, and R⁶ can each be C₁-C₆ alkyl. In any of the disclosed formula, Z can be S. In preferred examples, Z can be O.

In another specific embodiment, disclosed are compounds having Formula V:

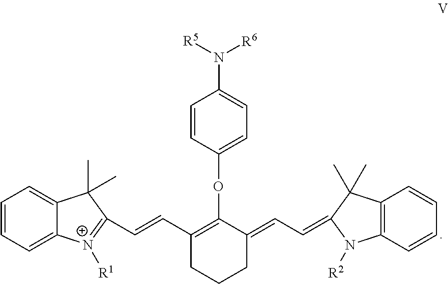

In any of these disclosed formula, R¹ and R² can be, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, R¹ and R² can be, independent of one another, alkyl substituted with sulfonic acid or sulfonate. In a preferred example, R¹ and R² can be C₁-C₆ alkyl. In any of these disclosed formula, R⁵ and R⁶ can be, independent of one another, alkyl, alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol. In other examples, R⁵ and R⁶ can be, independent of one another, alkyl optionally substituted with amine, amido, alkylester, carbonyl, carboxylate, carbamyl, or hydroxyl. Specific examples of compounds include:

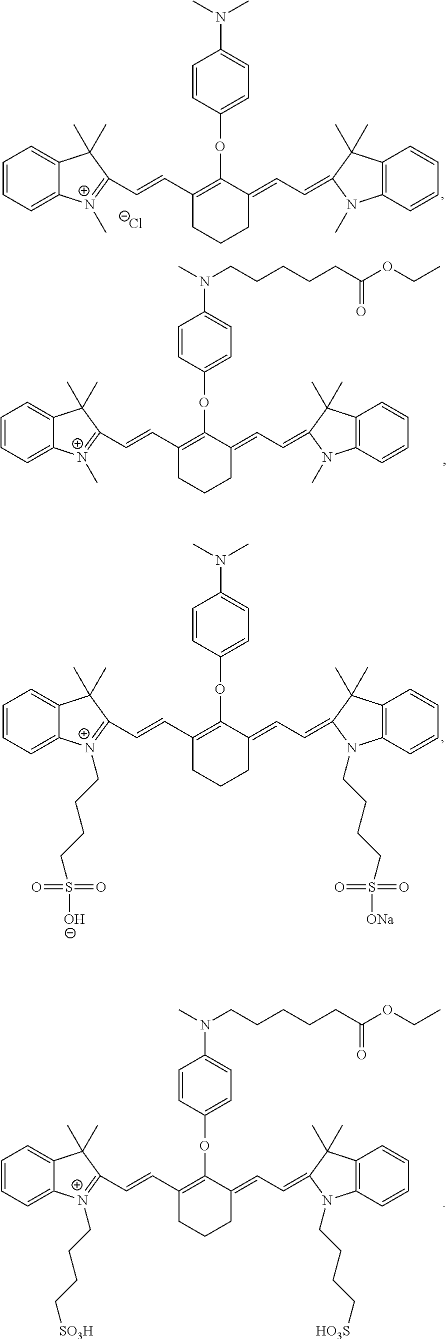

In any of the disclosed formula, the compound can be a chloride salt.

In other specific embodiments, disclosed herein are methods of detecting cancerous tissue in an individual, comprising: administering to the individual a compound having any of the formula disclosed herein and detecting a fluorescent signal from the compound. In specific examples, the compound can have Formula I, II, III, IV, or V. In certain examples, the compound can be administered by intraperitoneal injection. In certain examples, the compound can be administered topically. In certain examples, the compound can be administered before, during or after a tumor resection procedure. In certain examples, the compound can be applied to an excised tumor tissue to direct surgical procedure during surgery. In certain examples, the cancerous tissue can be ovarian cancer, colorectal cancer, brain cancer or breast cancer. In certain examples, the the cancerous tissue can be bladder cancer, cervical cancer, gastrointestinal cancer, genitourinary cancer, head and neck cancer, lung cancer, pancreatic cancer, prostate cancer, renal cancer, skin cancer, and testicular cancer. In a preferred example, the compound has Formula V.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims.

Further, while only certain representative compounds, methods, and aspects of these compounds and methods are specifically described, other compounds and methods and combinations of various features of the compounds and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

1. A pH sensitive fluorescent compound having Formula V: 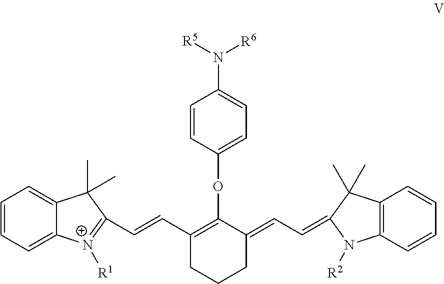
wherein
R¹ and R² are, independent of one another, H or alkyl, alkenyl, alkynyl, cycloalkyl, heterocycloalkyl, cycloalkenyl, or heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol; and
R⁵ and R⁶ are, independent of one another, H alkyl, alkenyl, alkynyl, cycloalkyl, heterocycloalkyl, cycloalkenyl, heterocycloalkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol;
or a pharmaceutically acceptable salt thereof.

2. (canceled)

3. (canceled)

4. The compound of claim 1, wherein R¹ and R² are, independent of one another, alkyl or alkenyl, either of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol.

5. The compound of claim 1, wherein R¹ and R² are, independent of one another, alkyl substituted with sulfonic acid or sulfonate.

6. The compound of claim 1, wherein R¹ and R² are C₁-C₆ alkyl.

7. The compound of claim 1, wherein R⁵ and R⁶ are, independent of one another, alkyl or alkenyl, any of which are optionally substituted with sulfonic acid, sulfonate, amino, amido, alkyl, alkenyl, alkynyl, alkoxyl, aryl, carbonyl, carboxylate, carbamyl, cyano, ester, halogen, heteroaryl, hydroxyl, nitrile, nitro, sulfinyl, sulfanyl, or thiol.

8. The compound of claim 1, wherein R⁵ and R⁶ are, independent of one another, alkyl optionally substituted with amine, amido, alkylester, carbonyl, carboxylate, carbamyl, or hydroxyl.

9. The compound of claim 1, wherein R⁵ and R⁶ are both be C₁-C₆ alkyl.

10. The compound of claim 1, wherein R¹, R², R⁵, and R⁶ are each C₁-C₆ alkyl.

11-19. (canceled)

20. A compound of claim 1, wherein the compound has one of the following formulas:

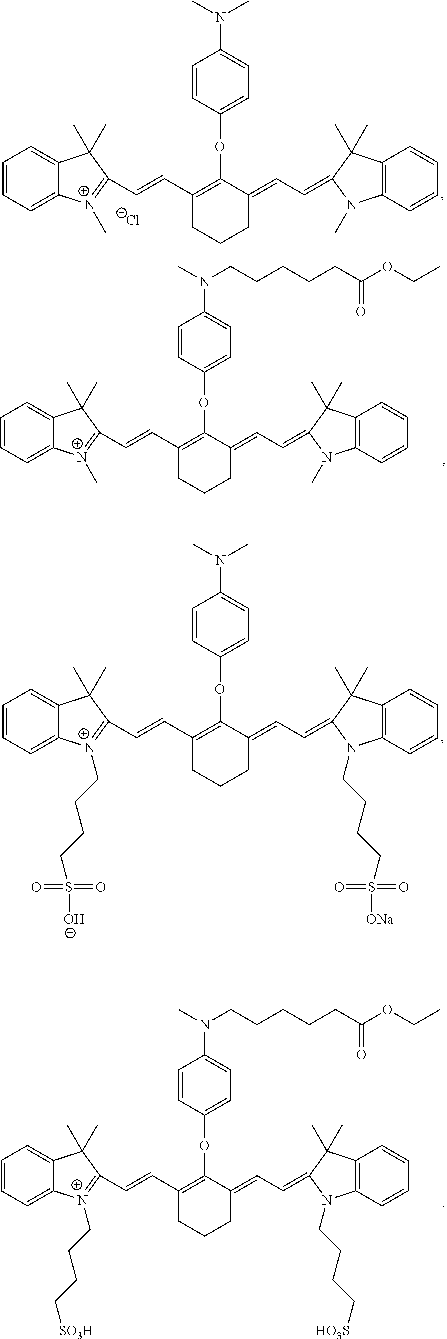

21. A formulation comprising the compound claim 1 and a pharmaceutically acceptable solvent.

22. A method of detecting cancerous tissue in an individual, comprising: administering to the individual a compound of claim 1 and detecting a fluorescent signal.

23. The method of claim 22, wherein the compound or formulation is administered by intraperitoneal injection.

24. The method of claim 22, wherein the compound or formulation is administered topically.

25. The method of claim 22, wherein the compound or formulation is administered by spraying the compound or formulation onto the individual.

26. The method of claim 22, wherein the compound or formulation is administered before, during or after a tumor resection procedure.

27. The method of claim 22, wherein the compound or formulation is applied to an excised tumor tissue to direct surgical procedure during surgery.

28. The method of claim 22, wherein the cancerous tissue is ovarian cancer, colorectal cancer, brain cancer or breast cancer.

29. The method of claim 22, wherein the cancerous tissue is bladder cancer, cervical cancer, gastrointestinal cancer, genitourinary cancer, head and neck cancer, lung cancer, pancreatic cancer, prostate cancer, renal cancer, skin cancer, and testicular cancer.

30. The method of claim 22, wherein the fluorescence signal is detected within 5 minutes of administering the compound or formulation.
